(19) United States
(12) Patent Application Publication
(10) Pub. No.: US 2016/0369318 A1
(43) Pub. Date: Dec. 22, 2016

(54) METHOD OF DETERMINING THE CONCENTRATION OF PATHOGENS OR OXIDIZABLE ORGANIC COMPOUNDS USING AN OZONE TITRATION SENSOR

(71) Applicant: Advanced Diamond Technologies, Inc., Romeoville, IL (US)

(72) Inventors: John Arthur Carlisle, Plainfield, IL (US); Donato M. Ceres, Chicago, IL (US); John Wagner, Hawthorn Woods, IL (US); John d. Yerger, III, Hawthorn Woods, IL (US)

(73) Assignee: Advanced Diamond Technologies, Inc., Romeoville, IL (US)

(21) Appl. No.: 15/189,380

(22) Filed: Jun. 22, 2016

Related U.S. Application Data
(60) Provisional application No. 62/183,145, filed on Jun. 22, 2015.

(51) Int. Cl. C12Q 1/04; G01N 31/16

(52) U.S. Cl. CPC C12Q 1/04; G01N 31/16

(57) ABSTRACT

The invention describes a method of ozone titration sensing which utilizes an ozone addition to a target solution, detection of ozone using an Oxidation-Reduction Potential (ORP) electrode or an Ultraviolet (UV) absorption photodiode or other means to detect ozone and the determination of the relative concentration of organics or pathogens subject to ozone oxidation which are present in the target solution. The inventive sensing method can be usefully employed to determine the relative concentration of pathogens such as viruses, bacteria and/or parasites that are readily oxidizable by ozone in aqueous solutions. The inventive sensing method may be used to control an ozone (or other oxidizing or disinfecting) compound dispensing system to optimize the dosage of ozone (or other disinfecting compound) necessary to produce a desired kill ratio or to generate a desired residual of ozone concentration in an aqueous solution after pathogen disinfection.

This application claims the benefit of U.S. Provisional Application Ser. No. 62/183,145 filed on Jun. 22, 2015. The entire contents of the Provisional Application are incorporated herein by reference for all purposes.

### FIELD OF THE INVENTION

This invention relates to a method for the determination of the concentration of pathogens and/or oxidizable organic compounds using ozone as a titrant. The technology can be used for either rapid real-time monitoring of the quality of water entering into a facility (i.e., business, school, or hospital), or POE (Point Of Entry), or to provide a means to alert a user that the quality of the water coming out of a faucet or other similar plumbing fixture (shower head, etc.) has diminished in real-time, or as a means of ensuring that a water treatment product that is designed to continuously clean water coming out of a faucet or similar fixture is working normally for POU (point of use) applications. The invention is not limited to any particular technology to generate and sense ozone dissolved in water, and can be tailored to meet the needs of specific applications.

### BACKGROUND

Current practice in the U.S. and elsewhere is to treat potable water in large centralized facilities and rely on the persistence of biocides such as chlorine to maintain the quality of the water as it transits from centralized facilities through the distribution system to end-customers. Recently there have been several incidents in which the system has broken down and people have been exposed to pathogens harmful to human life (e.g. legionella, rotavirus, cryptosporidium, giardia) or harmful organic toxics (chemicals such as estradiol) or normal organic matter (incidents with rainfall that overwhelm water handling systems). No technologies exist today that can selectively detect, in real time, the presence of biological pathogens, and even measurements of normal organic matter, such as carbon-oxygen demand (COD), are based on laboratory analytical tools that are not capable of providing immediate alerts of sudden decreases in water quality. For hospital applications in particular, legionella outbreaks are of particular concern, yet currently very few hospitals routinely screen for bacterial pathogens that may be present in the water, and only do so today with tests that take hours to days to complete.

Also, among warfighters and first-responders there has been great concern regarding the potential for terrorist attacks that use weaponized forms of pathogens (E coli. H157), introduced into local water supplies, as a means of carrying out terrorist attacks. There is a clear need for a real-time means of detection for these pathogens and toxic chemicals that is robust, relatively inexpensive, and also able to monitor water quality in-line without rendering it non-potable.

### DESCRIPTION OF THE RELATED ART

Ozone gas dissolved in water (Ozonated water) has been used for over 100 years to treat water at large scales, and is a very well-studied biocide. Compared to other commonly used biocides, ozonated water has two primary advantages: 1) its effectiveness is far superior to most other oxidants in terms of the rate at which ozone inactivates pathogens. The CT (contact time, which is the rate of inactivation at a given concentration) times for ozone are typically orders of magnitude better than chlorine, chlorine dioxide, bromine, or peroxide, and 2) the disinfection by-products generated by ozone are generally far less problematic than for halogen-based oxidants, (i.e. no chlorinated or brominated toxic byproducts such as trihalomethanes, haloacids and other halocompounds) that are sometimes more toxic than the organics present in a solution before treatment. Also, the half-life of ozone in water is typically on the order of 10-20 min, since it spontaneously decomposes to form dissolved O₂. Thus, much less ozone is required in order to achieve the same degree of inactivation of pathogens as compared to other disinfectant chemicals (typically chlorine-based), and the disinfected water that results contains fewer toxic by-products. The exception to this is in the relatively rare case in which the water contains a high concentration of bromine, in which case ozone reacts to form bromate which is toxic and tightly regulated.

Ozone in water is a non-selective biocide, which means that it will react with most forms of organic matter including bacterial, viral, and cyst-based pathogens, such as Cryptosporidium and Giardia, as well as many toxic or unwanted chemicals including hormones and Endocrine Disrupting Chemicals (EDCs). There are some exceptions, such as phenolic compounds and fluorinated hydrocarbons such as freons, but these chemicals are not commonly the source of concern for most potential target customers. Ozone reactions with hydrocarbons and other carbon-containing compounds typically proceed according to the following overall formula:

CₙH₂ₙ₊₂+4nO₃→nCO₂+(n+1)H₂O+4nO₂ Equation 1

Therefore for a mass of a hydrocarbon composed mostly of carbon and hydrogen to be completely oxidized with ozone the mass ratio of ozone required would be roughly (4×48)/15 or an approximate net mass ratio of 13:1 (i.e., a given mass of ozone-oxidizable organic would require an ozone mass of roughly 13 times the mass of organic being oxidized). This mass ratio is roughly 3 times the COD value for a given target oxidizable organic (or pathogen) load.

The US EPA definition of clean water includes a residual FAC (Free and Available Chlorine) level of greater than 0.1 ppm as delivered at the end of the distribution system, i.e. a home faucet. Through use of a real-time FAC measurement, it is possible to determine if water meets this definition. Unfortunately, many pathogens and toxic chemicals are resistant to chlorine. Ozone, having a much higher oxidation potential, i.e. a half cell potential (Eₒ)=+2.07V, (highest of any commonly used oxidant) as compared to Cl₂(aq) Eₒ=+1.36V, reacts with all pathogens and all but the most refractory chemicals. Thus, a more rigorous definition of water free of harmful organic matter can be made using an ozone residual in a similar manner to the FAC residual commonly used today. In effect, ozone in water can be considered the ultimate titrant that can be generated and used as such in real-time to monitor the quality of the water. Ozone in water can be detected in real-time using several technologies, including UV-absorption, electrochemical detection, and via use of an ORP (oxidation reduction potential) measurement. ORP is in fact the basis for defining water quality in most of the world today and is a relatively inexpensive technology.

Ozone can be generated using several methods, including UV-light, corona discharge, and electrochemistry. Corona discharge is the most commonly used approach and is well suited for large-scale generation of ozone, but it not optimal for smaller scale applications such as the sensing application that is the focus of the present invention. UV can be used to generate smaller concentrations but it is not sufficiently reliable to properly enable the invention. Electrochemistry is well suited for the real-time generation of ozone directly in water, but in the past has been greatly hindered by the toxicity and unreliability of PbO₂ and Pt electrodes at the high cell voltages and current densities necessary for ozone generation, since they also dissolve in the target solutions as toxic Pb²⁺ or Pt⁺ ions. Both of these electrodes have high operating costs since they dissolve very quickly at the high cell voltages required for ozone generation and must be replaced regularly in normal use. Diamond film coated electrodes (anodes) utilized in electrochemical cells have emerged in the past several years as the preferred choice to generate ppm-level concentrations of ozone from potable water sources, and they can be directly integrated into common fixtures such as residential and commercial faucets, shower heads, scrub stations, and other similar applications.

### SUMMARY OF THE INVENTION

The present invention describes a method of utilizing ozone to oxidize harmful pathogens such as bacteria, viruses or protozoa and oxidizable organic compounds and to use the detection of the quantity of ozone utilized for this purpose in a given volume of water or other solvent as a measure of the quantity of pathogen or oxidizable organic present in the solution. Ozone may be added to a sample solution and allowed to react for a specified time or distance from a point of addition. The ozone concentration remaining after the reaction time can be then be measured using an ozone detector. More ozone may be added to the same sample or a higher ozone concentration may be added to another portion of the same target sample and the resulting ozone concentration after reaction can be then be measured. A series of ozone additions to a given sample or a series of higher ozone concentrations to the aliquots of the same target sample may be used to generate a titration curve relating the concentration of residual ozone to the total dosage of ozone added to the sample (or aliquots of the same sample). A standard solution, which is preferably a sample of pure water, may be used to calibrate the ozone sensor and the ozone concentration resulting from addition of ozone to a given volume of target solution without reaction of the ozone with any pathogens or oxidizable organics. The ozone concentration resulting from addition of ozone to the target solution may be compared directly to the concentration resulting from the same quantity or concentration of ozone to the standard. Alternatively, the ratio of the ozone concentration measured in the target sample may be divided by the measured concentration of ozone from the standard to potentially permit a more accurate measure of the endpoint, i.e. the actual amount of ozone required to oxidize all the pathogens or oxidizable organics in the target solution. The relative concentration of pathogens or oxidizable organics may be calculated from the concentration of ozone required in a similar manner to the determination of the Chemical Oxygen Demand (COD) for wastewater.

### BRIEF DESCRIPTION OF THE DRAWINGS

FIG. 1 is a schematic representation of the inventive method where continuous or intermittent sampling of a target solution is shown to determine the concentration of pathogens and/or oxidizable organics.

FIG. 2 is a schematic representation of the inventive method where a standard or “blank” solution is also shown with ozone addition to allow a “zero” comparison of the amount of ozone present in the solution.

FIG. 3 is a graph of simulated ozone concentration data for both the target solution and for the standard solution using the inventive method of FIG. 2. The data is representative of ozone concentration data that would be obtained from an organic concentration reacting with two relative units of ozone.

FIG. 4 is a graph of simulated ozone concentration data for both the target solution and the standard where the concentration data from the target solution is divided by the concentration data from the standard. The relative concentration shown is the same as that shown in FIG. 3, i.e. 2 relative concentration units.

### DETAILED DESCRIPTION

The invention describes a method of ozone titration sensing which utilizes an ozone addition to a target solution, detection of ozone using an Oxidation-Reduction Potential (ORP) electrode or an Ultraviolet (UV) absorption photodiode or other means to detect ozone and the determination of the relative concentration of organics or pathogens subject to ozone oxidation which are present in the target solution. A predetermined quantity of ozone titrant is added to a portion of a target solution to be analyzed after which the concentration of ozone in solution is measured by ORP or by UV absorption or other ozone detection means. After calibration of the detection means by standard addition of ozone to a pure solution containing no oxidizable organics or pathogens, the measurement of the decline in ozone concentration can be used to determine the concentration of ozone in the target solution. The measurement of ozone in a target solution can be performed in tandem with a measurement of ozone in a pure reference solution and the signal of the two solutions can be divided to determine the ozone concentration in the target solution relative to the standard. The measurements can be performed continuously to determine the concentration of ozone as a function of time. The quantity of added ozone can be adjusted as necessary to detect differing concentrations of target organics or pathogens. In addition, multiple detectors can be used at varied distances and path lengths from the point of addition to determine the ozone concentration as a function of time. Also, the measurement of ozone concentration can be performed on a small portion of a continuous stream of a target solution or a larger volume. The ozone being added by the sensor may be generated by conventional corona discharge or by electrochemical means including on diamond anodes. The inventive sensing method can be usefully employed to determine the relative concentration of pathogens such as viruses, bacteria and/or parasites that are readily oxidizable by ozone in aqueous solutions. The time dependence of the decline in ozone concentration can be used to estimate the relative concentration of small pathogens such as bacteria or viruses as compared to other larger organic compounds or refractory organics. Finally, the inventive sensing method may be used to control an ozone (or other oxidizing or disinfecting) compound dispensing system to optimize the dosage of ozone (or other disinfecting compound) necessary to produce a desired kill ratio or to generate a desired residual of ozone concentration in an aqueous solution after pathogen disinfection.

Ozone can be generated by any of the means described above as long as the ozone generated is effectively dissolved in an aqueous solution. Corona discharges generate ozone in a gaseous state and it must be solubilized in order to be effective in the oxidation of dissolved organics and/or pathogens carried in a solution. A given quantity of ozone generated by a Corona discharge must therefore be discounted by the solubility factor for the solubilization process. On the contrary, ozone generated electrochemically in an aqueous solution, is produced in a soluble form and therefore the dissolution efficiency is nearly 100%. Therefore, a preferred embodiment of the inventive utilizes electrochemical means of generating ozone, and in particular a doped diamond anode for an electrochemical cell operating at a current density of 1-2 A/cm². Such a current density would dissolve a PbO₂ anode in minutes and a Pt anode in days or weeks, while a doped diamond anode produced using the method developed at ADT would last many months to several years. The present application claims priority U.S. provisional patent application No. 62/173,504, applied for by Advanced Diamond Technologies with a priority date of Jun. 10, 2015, which describes a high reliability composite diamond electrode capable of operating at a current density of 1 A/cm² or greater for 10 years or more without failure.

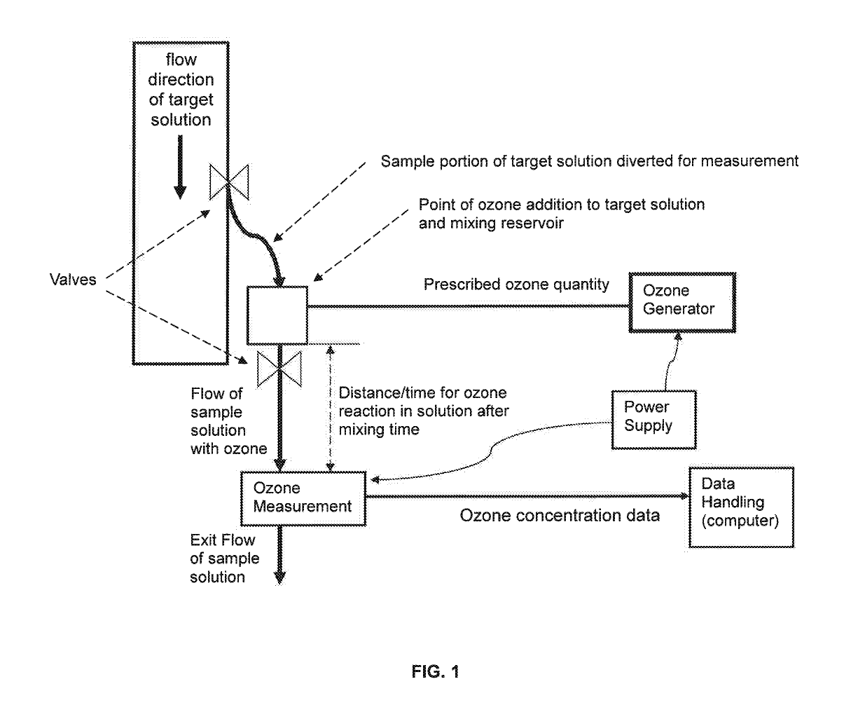

A schematic representation of an embodiment of the method presented herein is shown in FIG. 1. In FIG. 1, a small portion of the target solution is diverted for analysis by the inventive ozone titration method using the valve as shown. A prescribed amount of ozone is added to the target solution after it is generated and solubilized by one of the ozone generation means described above, e.g. corona discharge+solubilization, electrochemical, or UV photo-generation plus solubilization. After mixing with the portion of the target solution for a prescribed time duration, the remaining ozone concentration in solution is measured by one of the ozone measurement techniques described above, e.g. ORP electrode, UV absorption or other means. The time duration for reaction may be calculated or measured by the flow rate of the target solution after the point of addition towards the point of measurement. For example, a flow rate of 1 meter/sec through a tube would allow a reaction time of 1 second for a flow distance between the point of addition and the ozone measurement point of 1 meter.

The addition of an ozone concentration to the portion of the target solution may be via a series of additions to the given portion of the target solution or it may in a series of different concentrations in a descending or ascending quantity to same or similar concentration and volume aliquots of the same target sample. This “titration” of the target sample with varying quantities of ozone is performed to more accurately determine the concentration of pathogens or oxidizable organics in the solution.

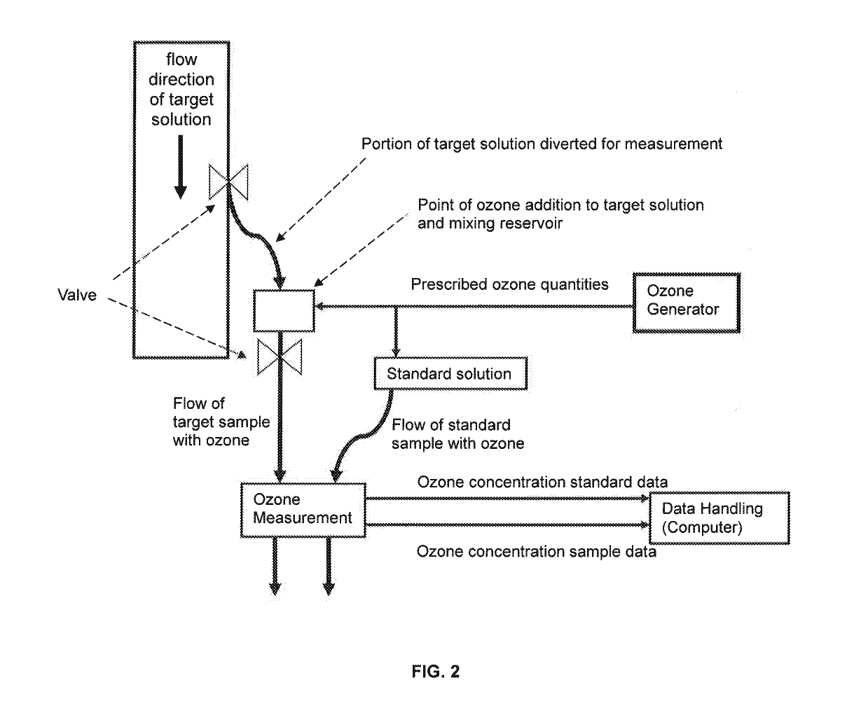

A similar configuration of hardware to accomplish the inventive method is shown in FIG. 2. FIG. 2 presents an additional loop containing a “standard” solution. In the case of aqueous solution, this will typically be pure water, without a significant quantity of dissolved organic compounds, i.e. COD ˜0, or any significant quantity of pathogens. Distilled water would usually be sufficient for this application. It is not necessary to use the sample volume of the standard as compared to the sample solution. However, the ozone dosage added to the standard as compared to the sample should be proportional to the volume ratio of the two. For example, if the standard volume is one tenth of the sample volume, the quantity (mass) of ozone added to the standard solution should be one tenth of the quantity added to the sample solution.

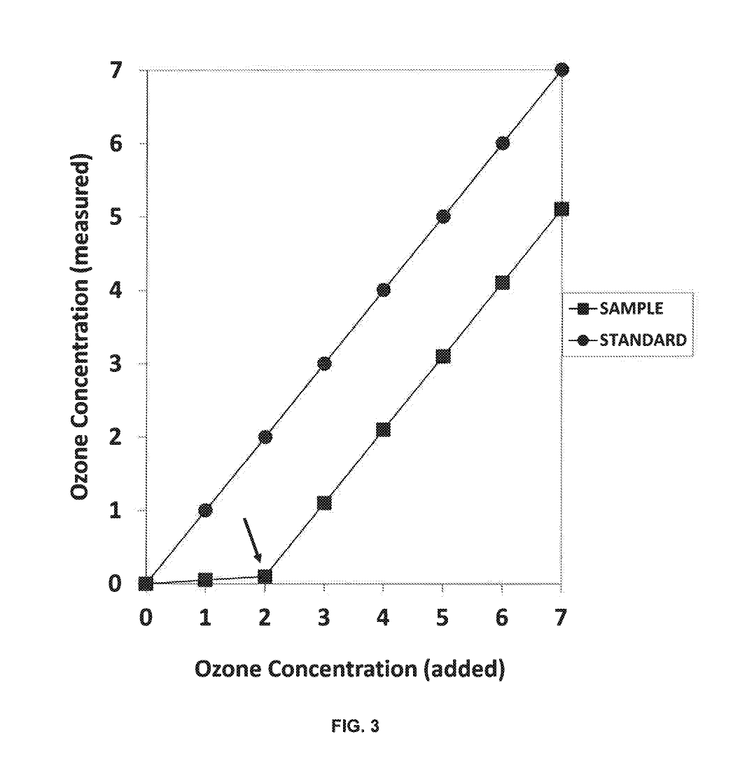

FIG. 3 presents simulated ozone concentration data for an approximate sample pathogen or oxidizable organic concentration of roughly 2 relative units of concentration of ozone (relatable to the organic concentration). Ozone added to the standard does not react due to the absence of oxidizable organics and/or pathogens, while ozone added to the target sample reacts up to the concentration of the oxidizable organics and/or pathogens present.

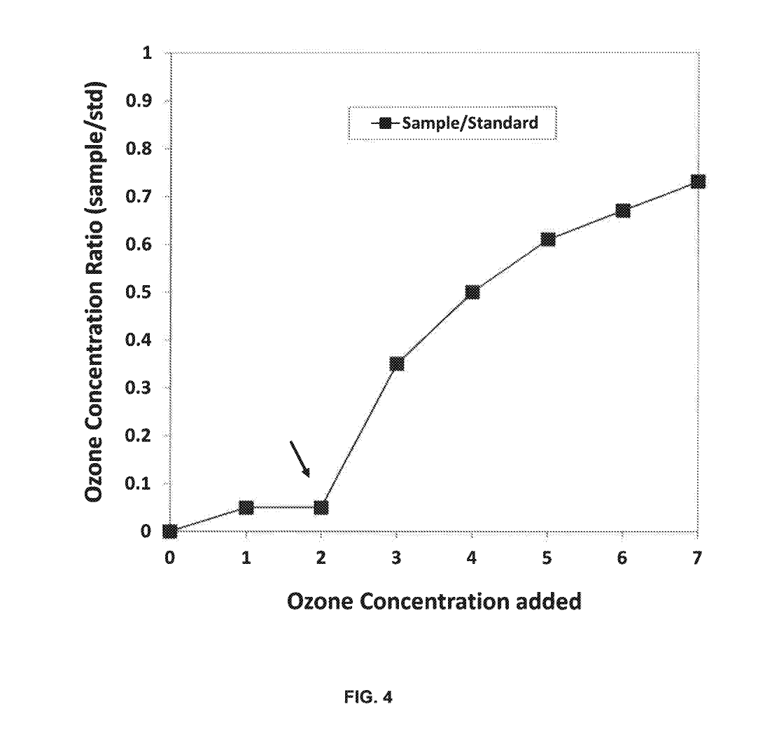

FIG. 4 presents simulated ozone concentration data for a ratio between the target solution ozone concentration and the standard ozone concentration for an approximate sample organic or pathogen concentration of roughly 2 relative units of concentration. Ozone added to the standard does not react due to the absence of oxidizable organics or pathogens, while ozone added to the target sample reacts up to the concentration of the oxidizable organics or pathogens present.

The following example will illustrate the inventive method in some detail using the configuration presented in FIG. 2. In this embodiment of the inventive method, a sample volume of 100 ml is diverted from the target solution and fluidically added to the sample reservoir. In this example, the portion of the target solution diverted to the reservoir contains 0.004 mg of humic acid (a typical dissolved organic compound found in surface waters that is readily oxidized by ozone) and 0.016 mg of bacteria and other pathogenic species for a total ozone oxidizable load of 0.2 mg/l (0.2 ppm). A first ozone dose of 1.3 mg could be added to the mixing reservoir. This could be accomplished, for example, by adding 130 ml of 10 mg/l (10 ppm) ozone to the mixing reservoir and allowing a few seconds for mixing and reaction of the ozone with the organics and pathogens in the solution. After this time, a small portion of the solution from the mixing reservoir, e.g. 1 ml, could be directed to the ozone detection system and the resultant ozone in the solution compared to the standard solution with the same overall concentration of ozone added to it. In this case, the standard would be required to have a 10 ppm ozone concentration diluted by 130/230, i.e. to 5.65 ppm. In this case, the standard would generate a concentration of 5.65 ppm when measured by the ozone measurement system. The sample solution, if properly mixed (e.g. after a few seconds with turbulent mixing using prior art methods), should generate a net ozone concentration of approximately zero since at a mass ratio of ˜13:1 for reaction of ozone with oxidizable organics, the 1.3 mg of ozone would oxidize roughly half of the 0.2 mg of organics in the target solution portion (i.e., the resultant concentration of ozone would be approximately zero and the resultant concentration of organics would be roughly 0.1 mg.

If a second point on a titration curve was required, an additional 1.3 mg of ozone could be added to the portion of the target solution in the mixing tank and allowed to react. This would then react with and destroy the remaining organics in the portion of the target solution resulting in a net ozone concentration and organic plus pathogen concentration of roughly zero. A third and subsequent points on the titration curve could be generated by another additions of the same quantity of ozone (1.3 mg) to generate a complete titration curve similar to the curve shown in FIG. 3 and a titration curve of the sample solution ozone concentration divided by the standard solution ozone concentration as shown in FIG. 4.

The preferred method outlined above is described in some detail to explain the method. In general such a titration method would be most effective in determining the concentration of pathogens and oxidizable organic contaminants in the solution accurately. However, for many applications, a more rapid and potentially less accurate single sample method would be sufficient and preferable. For such an example, and using the same sample concentration assumed above (i.e. 0.2 ppm), a single reading could be generated by selecting a small volume of this solution, e.g. 10 ml (i.e. with 0.002 mg of pathogens and oxidizable organic compounds) and adding an over-concentration of ozone, e.g. 0.13 mg of ozone and measuring the resultant concentration of ozone after mixing. If the measurements had been sufficiently characterized and calibrated with sample solutions of known concentration and ozone additions of known concentration, such a “one-off” measurement could be sufficient for many applications where a rapid approximate measure of the target solution's oxidizable pathogen and organic compound load is required. This would be particularly useful for rapid, POU measurements requiring continuous monitoring of a flowing source water with variable contaminant loads or for example a system requiring ongoing monitoring to control ozone additions or another oxidant to decontaminate a target.

The ozone sensing system outlined above and the resultant data on the concentration of oxidizable pathogens and organic compounds can be use used in order to control a system to decontaminate water. If a desired contaminant level is required, measurement of the contaminant level using the inventive method can be used as input data to determine point of use or ongoing dosing of decontamination chemicals or methods. For example, if the contaminant level were determined to be 0.2 ppm and the specification desired was close to zero, and if the contaminant (e.g. pathogens) was oxidizable by ozone, (which almost all pathogens are), the addition of 2.6 mg/l (2.6 ppm) of ozone could be effected downstream of the measurement system to decontaminate the solution. Higher ozone concentrations would be required for higher contaminant concentrations. Other methods, such as chlorination, or Reverse Osmosis could also be utilized (dependent upon the data generated by the inventive method). Given how quickly such data could be generated (e.g. as a fast or faster than once every second if required), rapid and precise control of a decontamination system could be effected using this data.

The operational cost of the inventive method and electrochemical ozone generated can be roughly estimated given some reasonable assumptions about the sample and the accuracy required. For example, if a sampling rate of 1 per minute was desired with a target solution volume of 10 ml per sample and a required ozone dosage of 10 ppm, it would require 1 mg/minute of ozone (0.144 g/day). At a current efficiency of 10% and a typical electrochemical cell voltage of 25V, this corresponds to a current of 0.067 A and a power consumption of 1.68 W. At an electricity price of 10 cents per kilowatt-hour this works out to a price of 0.4 cents of electricity per day ($1.46/year). At a current density of 1 A/cm² and an electrode cost of $10/cm² (this is not a quote but only a very rough estimate for illustration), and an electrode lifetime of 1 year (very conservative), the electrode replacement cost $0.67/year for a total combined (conservative) operating cost of ˜$2/year.

The inventive method can be used for the determination of contaminant concentrations in any solution in which ozone can dissolve and oxidize target contaminants. This would include aqueous solutions, but also alcohols and organic solvents that are to varying degrees subject to oxidation by ozone. However, the use of the standard calibration approach described above could be used to “zero out” this effect of solvent oxidation by ozone. Even aqueous solutions would suffer to small degree from decomposition of ozone to form dissolved O₂ in solution, since ozone has a half-life of ˜10-20 minutes. If measurement of the ozone were delayed, this effect could become significant since the ozone being measured would be subject to disappearance depending upon the time since generation. Therefore, it is preferable that the method be employed to generate ozone at the POU and for analysis of the target sample solutions within seconds or at most a minute or two from the time of generation. However, the standard calibration method described above would help to minimize any inaccuracies in the determination of organic concentration resulting from this issue and many other contamination issues. Therefore, the standard calibration method is a preferred method of conducting the inventive method.

It should be realized that the preferred method for the practice of the inventive method can be generalized using generally accepted methods to apply to many target solutions across a wide range of ozone concentrations and sample volumes.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the present invention as set forth in the appended claims.

What is claimed:

1. A method of determining the concentration of oxidizable organic compounds and/or pathogens in solution comprising the steps of:
a) generating a prescribed quantity of ozone using an ozone generating system;
b) delivering a prescribed quantity of ozone from the ozone generating system to a target solution containing oxidizable organic compounds and/or pathogens;
c) measuring the quantity of ozone remaining in the target solution after a prescribed time duration with an ozone measurement system;
d) calculating the quantity of ozone remaining after reaction in the target solution, wherein the quantity of ozone remaining in solution after the prescribed time duration is a function of the amount of ozone added to the target solution and the amount of ozone that has reacted with the oxidizable organic compounds and/or pathogens.

2. The method of claim 1, wherein the ozone generation system comprises an electrochemical cell.

3. The method of claim 2, wherein the electrochemical cell comprises a doped diamond anode.

4. The method of claim 1, wherein the ozone generation system comprises a corona discharge.

5. The method of claim 1, wherein the pathogens comprise bacteria, viruses or protozoa.

6. The method of claim 1, wherein the oxidizable organic compounds comprise reduced sulfur compounds, naphthenic acids, alkanes, alkenes or alkynes.

7. The method of claim 1, wherein the ozone measurement system comprises an Oxidation Reduction Potential (ORP) electrode wherein the ORP potential is a function of the concentration of ozone present in the target solution.

8. The method of claim 1, wherein the ozone measurement system an UV absorption system tuned to a wavelength of approximately 250 nm and wherein the absorption of UV is a function of the concentration of ozone present in the target solution.

9. The method of claim 1, additionally comprising a step of adding a prescribed quantity of ozone to a reference solution and measuring the concentration of ozone in the reference solution.

10. The method of claim 9, wherein the quantity of ozone added to the reference solution is the same as the quantity added to the target solution.

11. The method of claim 9, additionally comprising a step of dividing the measured concentration of ozone in the target solution by the measured concentration in the reference solution and calculating a ratio of the two concentrations.

12. The method of claim 1, additionally comprising a step of flowing the target solution from a POE of ozone to the target solution to the ozone measuring system.

13. The method of claim 12, wherein the distance between the POE of ozone to the target solution and the ozone measuring system and the flow velocity of the target solution is a function of the prescribed time duration.

14. The method of claim 12, additionally comprising a step of adding a second or more prescribed quantities of ozone to the target solution and determining the remaining concentration of ozone after this second or further prescribed quantities of ozone addition.

15. The method of claim 12, wherein the second or more prescribed quantities of ozone are delivered in ascending or descending amounts which are then calculated as part of a titration curve.

16. The method of claim of 12, wherein the second or more prescribed quantities of ozone are added to the target solution at differing flow velocities in order to generate a time-dependent calculation of the rate of reaction of the added ozone with oxidizable organics and/or pathogens.

17. The method of claim 1, wherein the target solution is an aqueous solution.

18. The method of claim 1, additionally comprising a step of adding a quantity of oxidizer to the target solution, wherein the quantity added is a function of the concentration of oxidizable organics and/or pathogens calculated.

19. The method of claim 18, wherein the oxidizer is ozone, chlorine, persulfate, hydrogen peroxide, or mixed oxidants,

20. The method of claim 1, wherein the prescribed quantity of ozone delivered to the target solution is from 0.1 to 10 parts per million.

21. The method of claim 1, wherein the prescribed time duration is between 0.1 and 100 seconds.

22. The method of claim 1, wherein the addition of ozone is performed repeatedly and optionally periodically and the detection of ozone is timed to correlate with the period of ozone addition.

23. The method of claim 1, additionally comprising a step of selecting a representative portion of the target solution for ozone addition.

24. The method of claim 23, wherein the representative portion of the target solution is selected from a flowing target solution and wherein the ozone detection means are downstream of the ozone addition point.

25. The method of claim 24, additional comprising a step of selecting the flow velocity of the flowing target solution and thereby adjusting the prescribed time duration between ozone addition and ozone measurement.

26. A device to measure the concentration of oxidizable organic compounds and/or pathogens in a target solution employing the method of claim 1.
